(19) United States
(12) Patent Application Publication
(10) Pub. No.: US 2009/0143375 A1
(43) Pub. Date: Jun. 4, 2009

(54) Tricyclic Lactam Derivatives, Their Manufacture and Use as Pharmaceutical Agents

(75) Inventors: Guy Georges, Habach (DE); Bernhard Goller, Penzberg (DE); Hans-Willi Krell, Penzberg (DE); Anja Limberg, Basel (CH); Ulrike Reiff, Penzberg (DE); Petra Rueger, Penzberg (DE); Matthias Rueth, Penzberg (DE); Christine Schuell, Penzberg (DE); Mark Stahl, Tuebingen (DE)

Correspondence Address: HOFFMANN-LA ROCHE INC.;PATENT LAW DEPARTMENT, 340 KINGSLAND STREET, NUTLEY, NJ, 07110, US

(73) Assignee: F. HOFFMANN-LA ROCHE AG, Basel (CH)

(21) Appl. No.: 12/084,531

(22) PCT Filed: Dec. 13, 2006
(86) PCT No.: PCT/EP2006/011998

§ 371 Date: May 5, 2008

(30) Foreign Application Priority Data
Dec. 15, 2005 (EP) 05027427.3

(51) Int. Cl. A61K 31/4188; C07D 487/04; A61P 29/00; A61P 9/00; A61P 35/00; A61P 25/00; A61K 31/5377

(52) U.S. Cl. 514/232.8; 548/302.1; 544/139; 514/394

(57) ABSTRACT

Objects of the present invention are the compounds of formula I

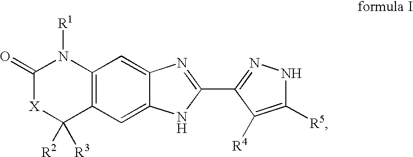

their pharmaceutically acceptable salts, enantiomeric forms, diastereoisomers and racemates, the preparation of the above-mentioned compounds, medicaments containing them and their manufacture, as well as the use of the above-mentioned compounds in the control or prevention of illnesses such as cancer.

The present invention relates to novel tricyclic lactam derivatives as protein kinase inhibitors, to a process for their manufacture, pharmaceutical compositions containing them and their manufacture as well as the use of these compounds as pharmaceutically active agents.

### BACKGROUND OF THE INVENTION

Protein kinases regulate many different signaling processes by adding phosphate groups to proteins (Hunter, T., Cell 50 (1987) 823-829); particularly serine/threonine kinases phosphorylate proteins on the alcohol moiety of serine or threonine residues. The serine/threonine kinase family includes members that control cell growth, migration, differentiation, gene expression, muscle contraction, glucose metabolism, cellular protein synthesis, and regulation of the cell cycle.

The Aurora kinases are a family of serine/threonine kinases that are believed to play a key role in the protein phosphorylation events that are essential for the completion of essential mitotic events. The Aurora kinase family is made up of three key members: Aurora A, B and C (also known as Aurora-2, Aurora-1 and Aurora-3 respectively). Aurora-1 and Aurora-2 are described in U.S. Pat. No. 6,207,401 of Sugen and in related patents and patent applications, e.g. EP 0 868 519 and EP 1 051 500.

For Aurora A there is increasing evidence that it is a novel proto-oncogene. Aurora A gene is amplified and transcript/protein is highly expressed in a majority of human tumor cell lines and primary colorectal, breast and other tumors. It has been shown that Aurora A overexpression leads to genetic instability shown by amplified centrosomes and significant increase in aneuploidy and transforms Rati fibroblasts and mouse NIH3T3 cells in vitro. Aurora A-transformed NIH3T3 cells grow as tumors in nude mice (Bischoff, J. R., and Plowman, G. D., Trends Cell Biol. 9 (1999) 454-459; Giet, R., and Prigent, C., J. Cell Sci. 112 (1999) 3591-3601; Nigg, E. A., Nat. Rev. Mol. Cell. Biol. 2 (2001) 21-32; Adams, R. R., et al., Trends Cell Biol. 11 (2001) 49-54). Moreover, amplification of Aurora A is associated with aneuploidy and aggressive clinical behavior (Sen, S., et al., J. Natl. Cancer Inst. 94 (2002) 1320-1329) and amplification of its locus correlates with poor prognosis for patients with node-negative breast cancer (Isola, J. J., et al., Am. J. Pathology 147 (1995) 905-911). For these reasons it is proposed that Aurora A overexpression contributes to cancer phenotype by being involved in chromosome segregation and mitotic checkpoint control.

Human tumor cell lines depleted of Aurora A transcripts arrest in mitosis. Accordingly, the specific inhibition of Aurora kinase by selective inhibitors is recognized to stop uncontrolled proliferation, re-establish mitotic checkpoint control and lead to apoptosis of tumor cells. In a xenograft model, an Aurora inhibitor therefore slows tumor growth and induces regression (Harrington, E. A., et al., Nat. Med. 10 (2004) 262-267).

Low molecular weight inhibitors for protein kinases are widely known in the state of the art. For Aurora inhibition such inhibitors are based on i.e. quinazoline derivatives (e.g. WO 00/44728), pyrimidine derivatives (e.g. WO 03/077921) imidazole, oxazole and thiazole derivatives (e.g. WO 02/96905 or WO 04/005283).

Aurora kinase inhibitors on the basis of pyrazole derivatives are described e.g. in WO 02/22601; WO 02/22602; WO 02/22603; WO 02/22604; WO 02/22605; WO 02/22606; WO 02/22607; WO 02/22608; WO 02/50065; WO 02/50066; WO 02/057259; WO 02/059111; WO 02/062789; WO 02/066461; WO 02/068415 or WO 2005/002552.

WO 03/035065 relates to benzimidazole derivatives as kinase inhibitors, especially as inhibitors against kinase insert domain containing receptor (KDR) tyrosine kinase, spleen tyrosine kinase (SYK) and inducible T cell kinase (ITK).

Some tricyclic compounds are known as inhibitors of erythrocyte aggregation from U.S. Pat. No. 4,835,280A and U.S. Pat. No. 4,954,498A. Also Mertens, A., et al., J. Med. Chem. 30 (1987) 1279-1287; von der Saal, W., et al., J. Med. Chem. 32 (1989) 1481-1491; U.S. Pat. No. 4,666,923A; U.S. Pat. No. 4,695,567A and U.S. Pat. No. 4,863,945A describe related tricycles as erythrocyte aggregation inhibitors. U.S. Pat. No. 5,212,186A describes tricycles for the treatment of cardiac insuffiency, hypertension and other diseases. WO 2006/032519 and WO 2006/063841 relate to pyrazolylbenzimidazole and tricyclic heterocycle imidazole derivatives as antitumor agents.

### SUMMARY OF THE INVENTION

The present invention relates to tricyclic aminopyrazole derivatives of the general formula I,

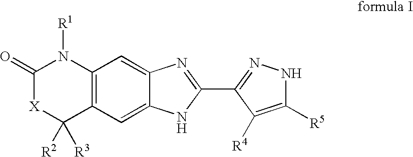

wherein,
R¹ is alkyl, which is substituted one or several times by halogen, nitro, cyano, hydroxy, amino, heterocyclyl, —C(O)OH, —C(O)NH₂ or —Y—R⁶; alkenyl, which is optionally substituted one or several times by halogen, nitro, cyano, hydroxy, amino, —C(O)OH, —C(O)NH₂ or —Y—R⁶; or
   alkynyl, which is optionally substituted one or several times by halogen, nitro, cyano, hydroxy, amino, —C(O)OH, —C(O)NH₂ or —Y—R⁶;
Y is —C(O)NH—, —C(O)N(alkyl)-, —N(alkyl)C(O)—, —NHC(O)—, —NHC(O)NH—, —NHC(O)N(alkyl)-, —NHS(O)₂—, —S(O)₂NH—, —S(O)₂N(alkyl)-, —S(O)₂—, —S(O)—, —C(O)O—, —OC(O)—, —C(O)—, —P(O)(alkyl)-, —NH—, —N(alkyl)-, —O— or —S—;
R⁶ is alkyl, wherein said alkyl is optionally substituted one or several times by halogen, hydroxy, alkoxy, alkoxyalkoxy, amino, alkylamino, dialkylamino, —C(O)OH or —C(O)NH₂;
   —(CH₂)ₙ-aryl, wherein the aryl is optionally substituted one or several times by halogen, cyano, nitro, amino, hydroxy, (C₁-C₄)alkyl, (C₁-C₄)alkoxy, halogenated (C₁-C₄)alkyl or halogenated (C₁-C₄)alkoxy; heteroaryl, wherein the heteroaryl is optionally substituted one or several times by alkyl;
   cycloalkyl; or
   heterocyclyl;
n is 0, 1 or 2;
R² is hydrogen or alkyl;
R³ is hydrogen or alkyl;
   or alternatively R² and R³ form together with the carbon atom to which they are attached a cycloalkyl ring;
R⁴ is hydrogen or alkyl;
R⁵ is hydrogen, alkyl, halogenated alkyl or cycloalkyl;
X is a single bond, —CH₂— or —C(alkyl)₂-;
and all pharmaceutically acceptable salts thereof.

The compounds according to this invention show activity as protein kinase inhibitors. Many diseases are associated with abnormal cellular responses triggered by protein kinase mediated events. These diseases include autoimmune diseases, inflammatory diseases, neurological and neurodegenerative diseases, cancer, cardiovascular diseases, allergies and asthma, Alzheimer's disease or hormone-related diseases. Accordingly, there has been a substantial effort in medicinal chemistry to find protein kinase inhibitors that are effective as therapeutic agents.

The compounds according to this invention in particular show activity as Aurora family kinase inhibitors, especially as Aurora A kinase inhibitors, and may therefore be useful for the treatment of diseases mediated by said kinase. Aurora A inhibition leads to cell cycle arrest in the G2 phase of the cell cycle and exerts an antiproliferative effect in tumor cell lines. This indicates that Aurora A inhibitors may be useful in the treatment of i.e. hyperproliferative diseases such as cancer and in particular colorectal, breast, lung, prostate, pancreatic, gastric, bladder, ovarian, melanoma, neuroblastoma, cervical, kidney or renal cancers, leukemias or lymphomas. Treatment of acute-myelogenous leukemia (AML, acute lymphocytic leukemia (ALL) and gastrointestinal stromal tumor (GIST) is included.

Objects of the present invention are the compounds of formula I and their tautomers, pharmaceutically acceptable salts, enantiomeric forms, diastereoisomers and racemates, their use as Aurora kinase inhibitors, the preparation of the above-mentioned compounds, medicaments containing them and their manufacture as well as the use of the above-mentioned compounds in treatment, control or prevention of illnesses, especially of illnesses and disorders as mentioned above like tumors or cancer (e.g. colorectal, breast, lung, prostate, pancreatic, gastric, bladder, ovarian, melanoma, neuroblastoma, cervical, kidney or renal cancers, leukemias or lymphomas) or in the manufacture of corresponding medicaments.

### DETAILED DESCRIPTION OF THE INVENTION

The term “alkyl” as used herein means a saturated, straight-chain or branched-chain hydrocarbon containing from 1 to 6, preferably 1 to 4, carbon atoms, such as methyl, ethyl, n-propyl, isopropyl, n-butyl, 2-butyl, t-butyl, n-pentyl, n-hexyl.

The term “alkenyl” as used herein means an unsaturated straight-chain or branched aliphatic hydrocarbon group containing one double bond and having 2 to 6, preferably 2 to 4 carbon atoms. Examples of such “alkenyl group” are vinyl (ethenyl), allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl, preferably allyl.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

The term “alkoxy” as used herein means an alkyl-O— group wherein the alkyl is defined as above. Examples include e.g. methoxy, ethoxy, isopropoxy, n-butoxy, 1-methyl-propoxy, 2-methyl-propoxy and the like.

The term “alkoxyalkoxy” as used herein means an alkyl-O-alkoxy group wherein alkyl and alkoxy are defined as above. Examples include e.g. 1-methoxy-ethoxy, 2-methoxy-ethoxy, 2-ethoxy-ethoxy, 2-propoxy-ethoxy, ethoxy-methoxy, methoxy-methoxy and the like.

The term “alkylamino” as used herein means an alkyl-NH— group wherein the alkyl is defined as above. Examples include e.g. N-methyl-amino, N-ethyl-amino, N-isopropyl-amino, N-(2-methyl-prop-1-yl)-amino and the like.

The term “dialkylamino” as used herein means an (alkyl)₂N— group wherein the alkyl is defined as above. Examples include e.g. N,N-dimethylamino, N-ethyl-N-methyl-amino, N,N-diethylamino and the like.

The term “alkyl, which is substituted one or several times by halogen, nitro, cyano, hydroxy, alkoxy, alkoxyalkoxy, amino, heterocyclyl, —C(O)OH, —C(O)NH₂ or —Y—R⁶” as used herein means an alkyl as defined above which is substituted one to six times, preferably one to three times by halogen, preferably by fluorine or chlorine, especially by fluorine, or which is substituted one to three times, preferably one to two times, especially one time by nitro, cyano, hydroxy, alkoxy, alkoxyalkoxy, amino, alkylamino, dialkylamino, —C(O)OH, —C(O)NH₂ or —Y—R⁶. Examples of such substituted alkyl groups are difluoromethyl, trifluoromethyl, 2,2,2-trifluoroethyl, perfluorethyl, difluoromethoxy, trifluoromethoxy, 2,2,2-trifluoroethoxy, perfluoroethoxy, 2-hydroxy-butyl, 2-hydroxy-ethyl, 2-hydroxy-propyl, 3-hydroxy-butyl, 2,3-dihydroxy-propyl, 2,3-dihydroxy-butyl, 1,2,3-trihydroxy-propyl, 2-hydroxy-pentyl, 2-methoxy-ethyl, 2-ethoxy-ethyl, 4-methoxy-butyl, 2-methoxy-butyl, 2-ethoxy-propyl, 3-propoxy-butyl, 2,3-dimethoxy-propyl, 2-ethoxy-3-methoxy-propyl, 2,3-diethoxy-butyl, 1,2,3-trimethoxy-propyl, 2-methoxy-pentyl, 2-(2-methoxy-ethoxy)-ethyl, 2-(2-ethoxy-ethoxy)-ethyl, 2-(2-propoxy-ethoxy)-ethyl, 3-(2-methoxy-ethoxy)-propyl, 3-(1-methoxy-ethoxy)-propyl, 4-(2-ethoxy-ethoxy)-butyl, 2-amino-butyl, 2-amino-ethyl, 2-amino-propyl, 3-amino-propyl, 3-amino-butyl, 2,3-diamino-propyl, 2-methylamino-butyl, 2-ethylamino-ethyl, 2-dimethylamino-ethyl, 2-dimethylamino-propyl, 3-diethylamino-propyl, 3-amino-butyl, 2,3-diamino-propyl, preferably 2,3-dihydroxy-propyl, 2-methoxy-ethyl, 2-(2-methoxy-ethoxy)-ethyl, trifluoromethyl, trifluoromethoxy.

The term “alkenyl, which is optionally substituted one or several times by halogen, nitro, cyano, hydroxy, alkoxy, alkoxyalkoxy, amino, alkylamino, dialkylamino, —C(O)OH, —C(O)NH₂ or —Y—R⁶” as used herein means an alkenyl as defined above which is optionally substituted one to six times, preferably one to three times by halogen, preferably by fluorine or chlorine, especially by fluorine, or which is optionally substituted one to three times, preferably one to two times, especially one time by nitro, cyano, hydroxy, alkoxy, alkoxyalkoxy, amino, alkylamino, dialkylamino, —C(O)OH, —C(O)NH₂ or —Y—R⁶.

The term “alkynyl, which is optionally substituted one or several times by halogen, nitro, cyano hydroxy, alkoxy, alkoxyalkoxy, amino, alkylamino, dialkylamino, —C(O)OH, —C(O)NH₂ or —Y—R⁶” as used herein means an alkynyl as defined above which is substituted one to six times, preferably one to three times by halogen, preferably by fluorine or chlorine, especially by fluorine, or which is optionally substituted one to three times, preferably one to two times, especially one time by nitro, cyano, hydroxy, alkoxy, alkoxyalkoxy, amino, alkylamino, dialkylamino, —C(O)OH, —C(O)NH₂ or —Y—R⁶.

The term “wherein the aryl is optionally substituted one or several times by” as used herein means that the aryl group in R⁶ is optionally substituted one to five times, preferably one to three times, especially one to two times.

The term “wherein the heteroaryl is optionally substituted one or several times by” as used herein means that the heteroaryl group in R⁶ is optionally substituted where possible one to two times, preferably one time.

The term “halogenated alkyl” as used herein means an alkyl group as defined above which is substituted one or several times, preferably one to six and especially one to three times, by halogen, preferably by fluorine or chlorine, especially by fluorine. Examples are difluoromethyl, trifluoromethyl, 2,2,2-trifluoroethyl, perfluorethyl, and the like, especially trifluoromethyl.

The term “halogenated alkoxy” as used herein means an alkoxy group as defined above which is substituted one or several times by halogen, preferably by fluorine or chlorine, especially fluorine. Examples are difluoromethoxy, trifluoromethoxy, 2,2,2-trifluoroethoxy, perfluoroethoxy and the like, especially trifluoromethoxy.

The term “cycloalkyl” means a monocyclic saturated hydrocarbon ring with 3 to 7, preferably 3 to 6, ring atoms. Such saturated carbocyclic groups can be optionally substituted one or several times, preferably one to three times by alkyl, especially one to two times. Preferably such saturated carbocyclic groups are unsubstituted. Examples of such saturated carbocyclic groups are cyclopropyl, cyclobutyl, cyclopentyl, cyclohexyl, cycloheptyl, 3-methyl-cyclopentyl, 3,3-dimethyl-cyclohexyl, 3-methyl-cyclohexyl, 2-methyl-cyclohexyl, preferably cyclopropyl.

The cycloalkyl ring which is formed by R² and R³ together with the carbon atom to which they are attached is preferably a cyclopentyl or cyclohexyl ring, especially a cyclopentyl ring. The cycloalkyl ring which is formed by R² and R³ together with the carbon atom to which they are attached is preferably a cyclopentyl or cyclohexyl ring, especially a cyclopentyl ring.

The term “heterocyclyl” means a saturated, monocyclic ring with 5 to 7 ring atoms which contains up to 3, preferably 1 or 2 heteroatoms selected independently from N, O or S and the remaining ring atoms being carbon atoms. Such saturated heterocyclic group can be optionally substituted one or several times, preferably one or two times a) by alkyl, preferably methyl, b) by —C(O)-alkyl, preferably acetyl, c) by oxo or d) by —S(O)₂-alkyl. Preferred substituents are a) alkyl or b) —C(O)-alkyl. Examples of such saturated heterocyclic groups include pyrrolidinyl, morpholinyl, piperazinyl, N-methyl-piperazinyl, N-acetyl-piperazinyl, piperazin-2-one, piperidyl, oxazolidine, thiazolidine, azepane and the like, preferably morpholinyl.

The term “aryl” means a mono- or bicyclic aromatic ring with 6 to 10 ring carbon atoms. Examples of such aryl groups are phenyl and naphthyl, preferably phenyl.

The term “heteroaryl” means a mono- or bicyclic aromatic ring with 5 to 10, preferably 5 to 6, ring atoms, which contains up to 3, preferably 1 or 2 heteroatoms selected independently from N, O or S and the remaining ring atoms being carbon atoms. Examples of such heteroaryl groups include pyrrolyl, imidazolyl, pyrazolyl, triazolyl, tetrazolyl, furanyl, oxazolyl, isoxazolyl, thienyl, thiazolyl, pyridyl, pyrimidyl, pyridazinyl, pyrazinyl, indolyl, indazolyl, benzimidazolyl, benzothiophenyl, benzofuranyl, quinolyl, isoquinolyl, quinazolinyl and the like, preferably pyridyl.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

As used herein, in relation to mass spectrometry (MS) the term “API+” refers to positive atmospheric pressure ionization mode, the term “API−” refers to negative atmospheric pressure ionization mode the term “ESI+” refers to positive electrospray ionization mode, the term “ESI−” refers to negative electrospray ionization mode.

As used herein, in relation to nuclear magnetic resonance (NMR) the term “D₆-DMSO” refers to deuterated dimethylsulfoxide.

The compounds of formula I can exist in different tautomeric forms and in variable mixtures thereof. All tautomeric forms of the compounds of formula I and mixtures thereof are an objective of the invention. For example, the imidazole part of the tricyclic ring system of formula I can exist in two tautomeric forms as shown here below:

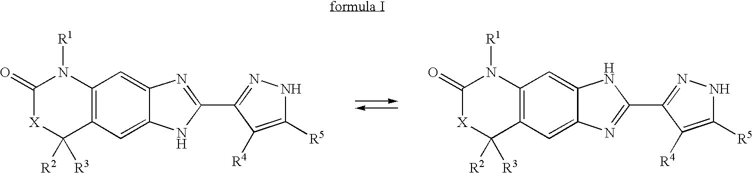

Also, e.g. the pyrazole ring of formula I can form two tautomeric forms as shown here below:

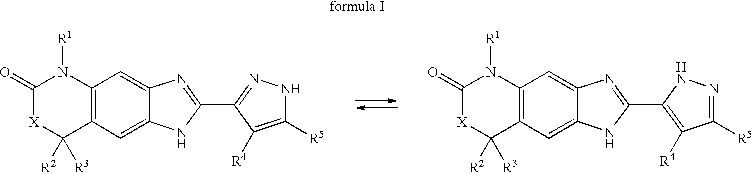

An embodiment of the invention are the compounds of formula I, wherein
R¹ is alkyl, which is substituted one or several times by halogen, nitro, cyano, hydroxy, amino, heterocyclyl, —C(O)OH, —C(O)NH₂ or —Y—R⁶; or
   alkenyl.

Another embodiment of the invention are the compounds of formula I, wherein
R¹ is alkyl, which is substituted one or several times by cyano, amino, heterocyclyl or —Y—R⁶; or
   alkenyl.

Another embodiment of the invention are the compounds of formula I, wherein
R¹ is alkyl, which is substituted one or several times by cyano, amino, heterocyclyl or —Y—R⁶.

Another embodiment of the invention are the compounds of formula I, wherein
R¹ is alkyl, which is substituted one or several times by —Y—R⁶.

Another embodiment of the invention are the compounds of formula I, wherein
R¹ is alkyl, which is substituted one or several times by cyano or amino

Such compounds, for example, may be selected from the group consisting of:
5-(2-Amino-ethyl)-7,7-dimethyl-2-(5-methyl-1H-pyrazol-3-yl)-5,7-dihydro-1H-imidazo[4,5-f]indol-6-one; and
[7,7-Dimethyl-2-(5-methyl-1H-pyrazol-3-yl)-6-oxo-6,7-dihydro-1H-imidazo[4,5-f]indol-5-yl]-acetonitrile.

Another embodiment of the invention are the compounds of formula I, wherein
R¹ is alkyl, which is substituted one or several times by heterocyclyl.

Such compounds, for example, may be selected from the group consisting of:
7,7-Dimethyl-5-(3-morpholin-4-yl-propyl)-2-(5-propyl-2H-pyrazol-3-yl)-5,7-dihydro-1H-imidazo[4,5-f]indol-6-one;
7,7-Dimethyl-2-(5-methyl-2H-pyrazol-3-yl)-5-(3-morpholin-4-yl-propyl)-5,7-dihydro-1H-imidazo[4,5-f]indol-6-one; and
7,7-Dimethyl-5-(3-morpholin-4-yl-propyl)-2-(5-trifluoromethyl-2H-pyrazol-3-yl)-5,7-dihydro-1H-imidazo[4,5-f]indol-6-one.

Another embodiment of the invention are the compounds of formula I, wherein
R¹ is alkenyl.

Such a compound, for example, may be selected from:
5-Allyl-7,7-dimethyl-2-(5-methyl-1H-pyrazol-3-yl)-5,7-dihydro-3H-imidazo[4,5-f]indol-6-one

Another embodiment of the invention are the compounds of formula I, wherein
R⁴ is hydrogen.

Another embodiment of the invention are the compounds of formula I, wherein
R² is hydrogen or alkyl;
R³ is hydrogen or alkyl;
R⁴ is hydrogen;
R⁵ is alkyl or halogenated alkyl; and
X is a single bond.

Another embodiment of the invention are the compounds of formula I, wherein
R² is hydrogen or alkyl;
R³ is hydrogen or alkyl;
R⁴ is hydrogen;
R⁵ is alkyl; and
X is a single bond.

Another embodiment of the invention are the compounds of formula I, wherein
Y is —C(O)NH—, —NHC(O)—, —NHC(O)NH—, NHS(O)₂—, —S(O)₂—, —S(O)—, —C(O)O—, —C(O)—, —NH—, —N(alkyl)-, —O— or —S—.

Another embodiment of the invention are the compounds of formula I, wherein
Y is —C(O)NH—, —C(O)O—, —C(O)—, —N(alkyl)- or —O—.

Another embodiment of the invention are the compounds of formula I, wherein
Y is —C(O)NH—.

Another embodiment of the invention are the compounds of formula I, wherein
Y is —C(O)—.

Another embodiment of the invention are the compounds of formula I, wherein
Y is —C(O)O—, —N(alkyl)- or —O—.

Another embodiment of the invention are the compounds of formula I, wherein
R⁶ is alkyl, wherein said alkyl is optionally substituted one or several times by halogen, hydroxy, alkoxy, alkoxyalkoxy, amino, alkylamino, dialkylamino, —C(O)OH or —C(O)NH₂.

Another embodiment of the invention are the compounds of formula I, wherein
R⁶ is alkyl.

Another embodiment of the invention are the compounds of formula I, wherein
R⁶ is —(CH₂)ₙ-aryl, wherein the aryl is optionally substituted one or several times by halogen or (C₁-C₄)alkoxy; and
n is 0 or 1.

Another embodiment of the invention are the compounds of formula I, wherein
R⁶ is heteroaryl, wherein the heteroaryl is optionally substituted one or several times by alkyl.

Another embodiment of the invention are the compounds of formula I, wherein
R⁶ is cycloalkyl.

Another embodiment of the invention are the compounds of formula I, wherein
R⁶ is heterocyclyl.

It will be understood that the above embodiments may be combined to form additional embodiments of the invention. Such combined embodiments are for example:

An embodiment of the invention are the compounds of formula I, wherein
R¹ is alkyl, which is substituted one or several times by cyano, amino, heterocyclyl or —Y—R⁶;
   or
   alkenyl;
Y is —C(O)NH—, —C(O)O—, —C(O)—, —N(alkyl)- or —O—;
R⁶ is alkyl;
   —(CH₂)ₙ-aryl, wherein the aryl is optionally substituted one or several times by halogen or (C₁-C₄)alkoxy;
   or heterocyclyl;
n is 0 or 1;
R² is hydrogen or alkyl;
R³ is hydrogen or alkyl;
R⁴ is hydrogen;
R⁵ is alkyl or halogenated alkyl; and
X is a single bond.

Another embodiment of the invention are the compounds of formula I, wherein
R¹ is alkyl, which is substituted one or several times by cyano, amino, heterocyclyl or —Y—R⁶;
Y is —C(O)NH—, —C(O)O—, —C(O)—, —N(alkyl)- or —O—;
R⁶ is alkyl;
   —(CH₂)ₙ-aryl, wherein the aryl is optionally substituted one or several times by halogen or (C₁-C₄)alkoxy;
   or heterocyclyl;
n is 0 or 1;
R² is hydrogen or alkyl;
R³ is hydrogen or alkyl;
R⁴ is hydrogen;
R⁵ is alkyl; and
X is a single bond.

Another embodiment of the invention are the compounds of formula I, wherein
R¹ is alkenyl;
R² is hydrogen or alkyl;
R³ is hydrogen or alkyl;
R⁴ is hydrogen;
R⁵ is alkyl; and
X is a single bond.

Another embodiment of the invention are the compounds of formula I, wherein
R¹ is alkyl, which is substituted one or several times by —Y—R⁶;
Y is —C(O)NH—, —C(O)O—, —C(O)—, —N(alkyl)- or —O—; and
R⁶ is alkyl.

Such compounds, for example, may be selected from the group consisting of:
[7,7-Dimethyl-2-(5-methyl-1H-pyrazol-3-yl)-6-oxo-6,7-dihydro-3H-imidazo[4,5-f]indol-5-yl]-acetic acid ethyl ester;
5-(2-Methoxy-ethyl)-7,7-dimethyl-2-(5-methyl-1H-pyrazol-3-yl)-5,7-dihydro-3H-imidazo[4,5-f]indol-6-one; and
5-(2-Diethylamino-ethyl)-7,7-dimethyl-2-(5-methyl-1H-pyrazol-3-yl)-5,7-dihydro-3H-imidazo[4,5-f]indol-6-one.

Another embodiment of the invention are the compounds of formula I, wherein
R¹ is alkyl, which is substituted one or several times by —Y—R⁶;
Y is —C(O)NH—, —C(O)O—, —C(O)—, —N(alkyl)- or —O—; and
R⁶ is —(CH₂)ₙ-aryl, wherein the aryl is optionally substituted one or several times by halogen or (C₁-C₄)alkoxy; and
n is 0 or 1.

Such compounds, for example, may be selected from the group consisting of:
N-Benzyl-2-[7,7-dimethyl-2-(5-methyl-1H-pyrazol-3-yl)-6-oxo-6,7-dihydro-3H-imidazo[4,5-f]indol-5-yl]-acetamide; compound with acetic acid;
2-[7,7-Dimethyl-2-(5-methyl-1H-pyrazol-3-yl)-6-oxo-6,7-dihydro-3H-imidazo[4,5-f]indol-5-yl]-N-(4-fluoro-phenyl)-acetamide;
N-(3,5-Dimethoxy-benzyl)-2-[7,7-dimethyl-2-(5-methyl-1H-pyrazol-3-yl)-6-oxo-6,7-dihydro-3H-imidazo[4,5-f]indol-5-yl]-acetamide; and
2-[7,7-Dimethyl-2-(5-methyl-1H-pyrazol-3-yl)-6-oxo-6,7-dihydro-3H-imidazo[4,5-f]indol-5-yl]-N-(4-fluoro-benzyl)-acetamide.

Another embodiment of the invention are the compounds of formula I, wherein
R¹ is alkyl, which is substituted one or several times by —Y—R⁶;
Y is —C(O)NH—, —C(O)O—, —C(O)—, —N(alkyl)- or —O—; and
R⁶ is heterocyclyl.

Such a compound, for example, may be selected from:
7,7-Dimethyl-2-(5-methyl-1H-pyrazol-3-yl)-5-(2-morpholin-4-yl-2-oxo-ethyl)-5,7-dihydro-3H-imidazo[4,5-f]indol-6-one.

The compounds of formula I may be prepared by any process known to be applicable to the preparation of chemically-related compounds. Such preparation is an object of the present invention.

One embodiment of the invention is a process for the preparation of the compounds of formula I, comprising the steps of:
a) reacting a compound of formula II

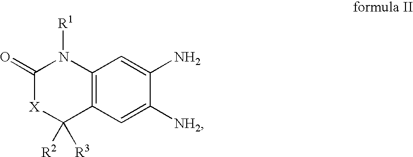

wherein R¹ to R³ and X have the significance given above for formula I;
with a compound of formula IV,

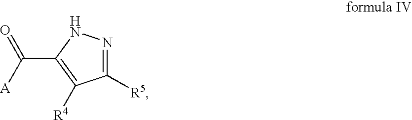

wherein A is —OH, —Cl, —H or —OMe and R⁴ and R⁵ have the significance given above for formula I;
to give the compounds of formula I,

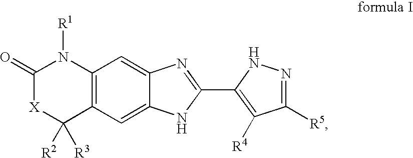

wherein R¹ to R⁵ and X have the significance given above for formula I;
b) isolating said compound of formula I is from the reaction mixture, and
c) if desired, converting said compound into a pharmaceutically acceptable salt or ester.

The compounds of formula I, or a pharmaceutically acceptable salt thereof, which are subject of the present invention, may be prepared by any process known to be applicable to the preparation of chemically-related compounds. Such processes, when used to prepare a compound of the formula I, or a pharmaceutically-acceptable salt thereof, are illustrated by the following representative schemes 1 to 3 and examples in which, unless otherwise stated, R¹, R², R³, R⁴, R⁵ and X have the significance given herein before for formula I. Necessary starting materials are either commercially available or they may be obtained by standard procedures of organic chemistry. The preparation of such starting materials is described within the accompanying examples or in the literature cited below with respect to scheme 1 to 3. Alternatively necessary starting materials are obtainable by analogous procedures to those illustrated which are within the ordinary skill of an organic chemist.

One route for the preparation of compounds of formula I starts from the diamines of formula II

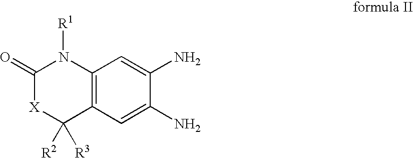

In formula II, X, R¹, R² and R³ have the significance as given above for formula I.

The synthesis of diamines of formula II or precursors thereof is described in Mertens, A., et al., J. Med. Chem. 30 (1987) 1279-1287; von der Saal, W., et al., J. Med. Chem. 32 (1989) 1481-1491; U.S. Pat. No. 4,666,923A, U.S. Pat. No. 4,695,567A, U.S. Pat. No. 4,863,945A and U.S. Pat. No. 4,985,448A. For instance, the diamines of formula II, wherein X is a single bond are named IIa and can be synthesized according to U.S. Pat. No. 4,666,923A, DE 34 10 168 and Mertens, A., et al., J. Med. Chem. 30 (1987) 1279-1287 as shown in Scheme 1a:

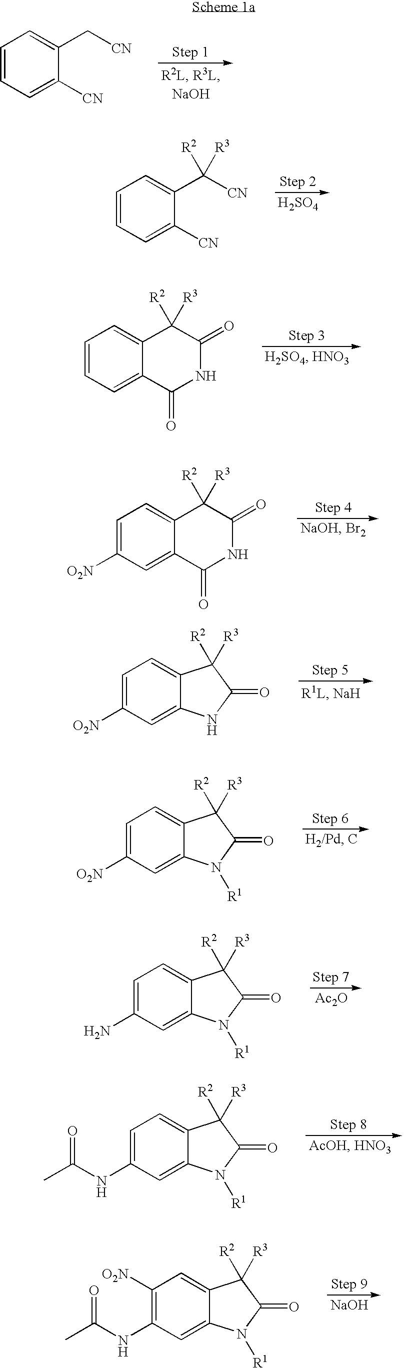

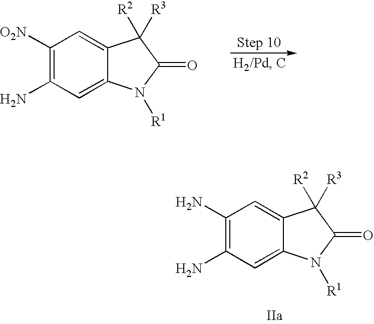

In scheme 1a, R¹, R² and R³ have the significance as given above for formula I, except that R¹ is not hydrogen, and L represents a leaving group as e.g. iodine, bromine, chlorine, triflate and the like.

In an alternative procedure diamines of formula II can be obtained by an alkylation of diamines of formula III as shown in scheme 1b. Diamines of formula III can be synthesized according to scheme 1 under omission of step 5.

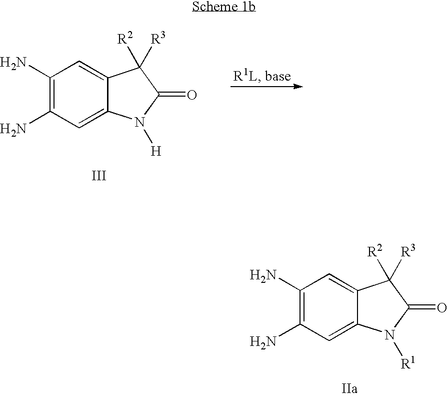

In scheme 1b, R¹, R² and R³ have the significance as given above for formula I, except that R¹ is not hydrogen, and L represents a leaving group as e.g. iodine, bromine, chlorine, triflate and the like. The alkylation reaction is typically carried out in the presence of a base such as sodium hydride, potassium hydride and the like, especially sodium hydride, in inert solvents such as dimethylformamide (DMF), N-methyl-pyrrolidinone (NMP), tetrahydrofuran and the like.

Diamines of formula II are subsequently employed in the formation of the imidazole ring system of formula I. Different synthetic pathways for this cyclization are described in the literature (e.g. see Mertens, A., et al., J. Med. Chem. 30 (1987) 1279-1287 and U.S. Pat. No. 4,695,567A).

For example, as shown in Scheme 2, diamines of formula II can be reacted with carboxylic acids (pyrazole compounds of formula IV wherein A is hydroxy), acid chlorides (pyrazole compounds of formula IV wherein A is chlorine), aldehydes (pyrazole compounds of formula IV wherein A is hydrogen), methyl carboxylates (pyrazole compounds of formula IV wherein A is methoxy) or activated esters (pyrazole compounds of formula IV wherein A is e.g. hydroxybenzotriazole). For detailed procedures see Mertens, A., et al., J. Med. Chem. 30 (1987) 1279-1287 and U.S. Pat. No. 4,695,567A.

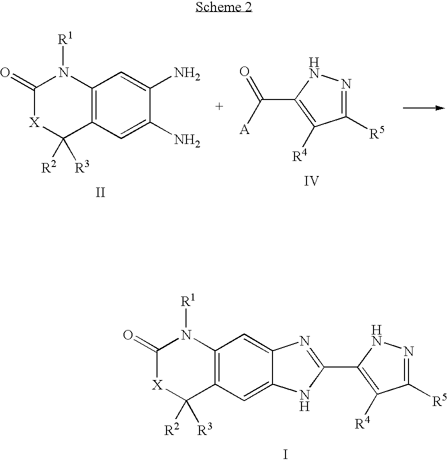

In scheme 2, R¹, R², R³, R⁵ and X have the significance as given above for formula I and A is hydroxy, chlorine, hydrogen, methoxy or e.g. hydroxybenzotriazole.

Pyrazoles of formula IV are commercially available or they can be prepared by standard procedures of organic chemistry (see e.g. Stanovnik, B., and Svete, J., Science of Synthesis 12 (2002) 15-225), e.g. condensation of a 1,3-dicarbonyl compound with hydrazine (see e.g. WO 04/032928 or van Herk, T., et al., J. Med. Chem. 46 (2003) 3945-3951) or 1,3-dipolar cycloaddition between a diazo compound and an acetylene (see e.g. Sewald, N., et al., Liebigs Ann. Chem. (1992) 947-952).

Pyrazoles of formula IV wherein R⁴ is hydrogen, R⁵ is trifluoromethyl and A is hydroxy can be prepared in a three step procedure according to Scheme 3: condensation of 4,4,4-trifluoro-1-(2-furyl)-1,3-butanedione with benzyl hydrazine under acidic conditions, oxidative degradation of the furan ring with potassium permanganate to the carboxylic acid functionality (see e.g. Djuric, S. W., et al., J. Med. Chem. 43 (2000) 2975-2981; Jia, Z. J., et al., Bioorg. Med. Chem. Lett. 12 (2002) 1651-1655 or Pruitt, J. R., et al., J. Med. Chem. 46 (2003) 5298-5315) and cleavage of the benzyl protecting group provides the desired 5-trifluoromethyl-2H-pyrazole-3-carboxylic acid.

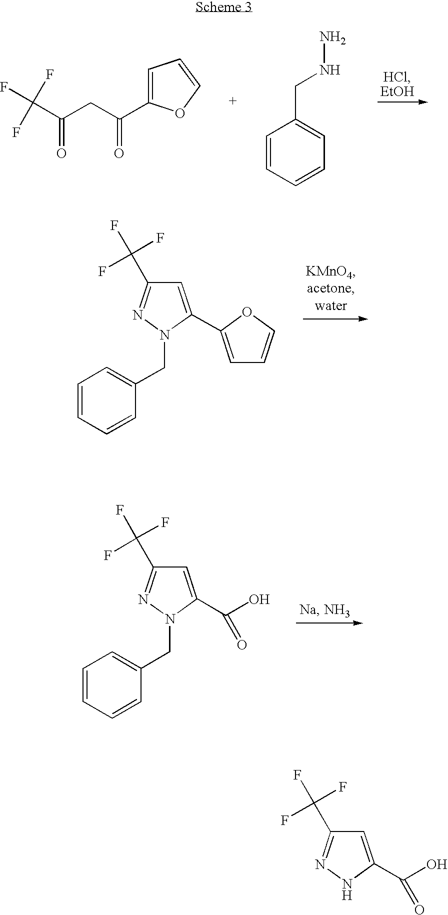

This procedure involving the N-benzyl or alternatively the p-methoxybenzyl group (Subramanyam, C., Synth. Commun. 25 (1995) 761-774) as intermediate protecting group can be also applied for preparing other pyrazoles needed as starting material.

Certain substituents on the groups R¹ may not be inert to the conditions of the synthesis sequences described above and may require protection by standard protecting groups known in the art. For instance, an amino or hydroxyl group may be protected as an acetyl or tert-butyloxycarbonyl (BOC) derivative. Alternatively, some substituents may be derived from others at the end of the reaction sequence. For instance, a compound of formula I may be synthesized bearing a nitro-, a cyano, an ethoxycarbonyl, an ether, a sulfonic acid substituent on the group R¹, which substituents are finally converted to an a) amino group—(e.g. by reduction of a nitro group, reduction of a cyano group or cleavage of a suitable amino protection group (for example by removal of a BOC group with trifluoroacetic acid (TFA))), b) alkylamino group—(e.g. by reductive amination of an amino group), c) dialkylamino group—(e.g. by alkylation of an amino group, reduction of an appropriate acylamino group with lithium aluminum hydride or Eschweiler-Clarke reaction with an appropriate amino or alkylamino group), d) acylamino group—(e.g. by amide formation from an amino group e.g. with appropriate acyl halides or with appropriate carboxylic acids after their activation with 1,1′-carbonyldiimidazole (CDI), 1-ethyl-3-[3-dimethylaminopropyl]carbodiimide hydrochloride (EDC), etc.), e) alkylsulfonylamino group (e.g. by reaction of an amino group with sulfonyl chlorides), f) arylsulfonylamino group substituent (e.g. by reaction of an amino group with sulfonyl chlorides), g) hydroxyl group—(e.g. by cleavage of a suitable hydroxy protection group (e.g. hydrogenolytic removal of a benzyl ether or oxidative cleavage of a p-methoxy benzyl ether or fluoride assisted cleavage of silyl protecting group), h) ether group—(e.g. by Williamson's ether synthesis from a hydroxyl group), i) carboxamide group (e.g. by amide formation from a carboxylic acid group with appropriate amines after activation of the carboxylic acid group with CDI, EDC, etc. or conversion to an acyl chloride), or j) sulfonamide group by standard procedures.

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g. Stahl, P. H., and Wermuth, G., (editors), Handbook of Pharmaceutical Salts, Verlag Helvetica Chimica Acta (VHCA), Zürich, (2002) or Bastin, R. J., et al., Organic Proc. Res. Dev. 4 (2000) 427-435.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases (HPLC: High Performance Liquid Chromatography) which are commercially available.

### Pharmacological Activity

The compounds of formula I and their pharmaceutically acceptable salts possess valuable pharmacological properties. It has been found that said compounds show activity as inhibitors of the Aurora kinase family and also show anti-proliferative activity. Consequently the compounds of the present invention are useful in the therapy and/or prevention of illnesses with known over-expression of kinases of the Aurora family preferably Aurora A, especially in the therapy and/or prevention of illnesses mentioned above. The activity of the present compounds as inhibitors of the Aurora kinase family is demonstrated by the following biological assay:

### IC₅₀ Determination for Inhibitors of Aurora A Kinase Assay Principle

Aurora A is a serine threonine kinase involved in spindle assembly and chromosome segregation.

The assay is a typically ELISA-type assay where substrate (GST-Histone H3) is coupled to the assay-plate and is phosphorylated by the kinase. Phosphorylation is detected by a mouse anti-Phosphopeptid mAb and an HRP-labeled anti-mouse pAb. The assay is validated for IC₅₀-determination.

Kinase activities were measured by Enzyme-Linked Immunosorbent Assay (ELISA): Maxisorp 384-well plates (Nunc) were coated with recombinant fusion protein comprising residues 1-15 of HistoneH3 fused to the N-terminus of Glutathione-S-Transferase. Plates were then blocked with a solution of 1 mg/mL I-block (Tropix cat# T2015—highly purified form of casein) in phosphate-buffered saline. Kinase reactions were carried out in the wells of the ELISA plate by combining an appropriate amount of mutant Aur A kinase with test compound and 30 μM ATP. The reaction buffer was 10× Kinase Buffer (Cell Signaling cat #9802) supplemented with 1 μg/mL I-block. Reactions were stopped after 40 minutes by addition of 25 mM EDTA. After washing, substrate phosphorylation was detected by addition of anti-phospho-Histone H3 (Ser 10) 6G3 mAb (Cell Signaling cat #9706) and sheep anti-mouse pAb-HRP (Amersham cat# NA931V), followed by colorimetric development with TMB (3,3′,5,5′-tetramethylbenzidine from Kirkegaard & Perry Laboratories). After readout of the absorbance, IC₅₀ values were calculated using a non-linear curve fit (XLfit software (ID Business Solution Ltd., Guilford, Surrey, UK))

TABLE 1
Results:

| Example No. | IC50 Aurora A kinase inhibition [μM] |
|---|---|
| 1 | 0.08 |
| 10 | 0.04 |
| 2, 8, 9, 11, 12, 13, 14 | 0.01-0.10 |

### Antiproliferative Activity

The activity of the present compounds as antiproliferative agents is demonstrated by the following biological assay:
CellTiter-Glo™ Assay in HCT 116 Cells

The CellTiter-Glo™ Luminescent Cell Viability Assay (Promega) is a homogeneous method of determining the number of viable cells in culture based on quantitation of the ATP present, which signals the presence of metabolically active cells.

HCT 116 cells (human colon carcinoma, ATCC-No. CCl-247) were cultivated in RPMI 1640 medium with GlutaMAX™ I (Invitrogen, Cat-No. 61870-010), 2.5% Fetal Calf Serum (FCS, Sigma Cat-No. F4135 (FBS)); 100 Units/ml penicillin/100 μg/ml streptomycin (=Pen/Strep from Invitrogen Cat. No. 15140). For the assay the cells were seeded in 384 well plates, 1000 cells per well, in the same medium. The next day the test compounds were added in various concentrations ranging from 30 μM to 0.0015 μM (10 concentrations, 1:3 diluted). After 5 days the CellTiter-Glo™ assay was done according to the instructions of the manufacturer (CellTiter-Glo™ Luminescent Cell Viability Assay, from Promega). In brief: the cell-plate was equilibrated to room temperature for approximately 30 minutes and than the CellTiter-Glo™ reagent was added. The contents were carefully mixed for 15 minutes to induce cell lysis. After 45 minutes the luminescent signal was measured in Victor 2, (scanning multiwell spectrophotometer, Wallac).

### Details:

1st. day:
Medium: RPMI 1640 with GlutaMAX™ I (Invitrogen, Cat-Nr. 61870), 5% FCS (Sigma Cat.-No. F4135), Pen/Strep (Invitrogen, Cat No. 15140).
HCT116 (ATCC-No. CCl-247): 1000 cells in 60 μl per well of 384 well plate (Greiner 781098, μClear-plate white)
After seeding incubate plates 24 h at 37° C., 5% CO₂
   2nd. Day: Induction (Treatment with Compounds, 10 Concentrations):

In order to achieve a final concentration of 30 μM as highest concentration 3.5 μl of 10 mM compound stock solution were added directly to 163 μl media. Then step e) of the dilution procedure described below, was followed.

In order to achieve the second highest to the lowest concentrations, a serial dilution with dilution steps of 1:3 was followed according to the procedure (a-e) as described here below:
a) for the second highest concentration add 10 μl of 10 mM stock solution of compound to 20 μl dimethylsulfoxide (DMSO)
b) dilute 8×1:3 (always 10 μl to 20 μl DMSO) in this DMSO dilution row (results in 9 wells with concentrations from 3333.3 μM to 0.51 μM)
c) dilute each concentration 1:47.6 (3.5 μl compound dilution to 163 μl media)
e) add 10 μl of every concentration to 60 μl media in the cell plate resulting in final concentration of DMSO: 0.3% in every well and resulting in 10 final concentration of compounds ranging from 30 μM to 0.0015 μM.
   Each compound is tested in triplicate.
   Incubate 120 h (5 days) at 37° C., 5% CO₂

### Analysis:

Add 30 μl CellTiter-Glo™ Reagent (prepared from CellTiter-Glo™ Buffer and CellTiter-Glo™ Substrate (lyophilized) purchased from Promega) per well,
shake 15 minutes at room temperature
incubate further 45 minutes at room temperature without shaking

### Measurement:

Victor 2 scanning multiwell spectrophotometer (Wallac), Luminescence mode (0.5 sec/read, 477 nm)
Determine IC50 using a non-linear curve fit (XLfit software (ID Business Solution Ltd., Guilford, Surrey, UK))

With all compounds a significant inhibition of HCT 116 cell viability was detected, which is exemplified by the compounds shown in Table 1.

TABLE 2
Results:

| Examples | IC50 HCT 116 [μM] |
|---|---|
| 6 | 1.24 |
| 9 | 5.83 |
| 1, 2, 3, 4, 5, 10, 11, 12, 13 | 0.1-5.0 |
| 14 | 5.0-15 |

Medicaments containing a compound of the present invention or a pharmaceutically acceptable salt thereof and a pharmaceutically acceptable carrier are an object of the present invention, as is a process for their production, which comprises bringing one or more compounds of the present invention and/or pharmaceutically acceptable salts and, if desired, one or more other therapeutically valuable substances into a galenical administration form together with one or more pharmaceutically acceptable carriers.

In accordance with the invention the compounds of the present invention as well as their pharmaceutically acceptable salts are useful in the control or prevention of illnesses. Based on their Aurora kinase inhibition and their antiproliferative activity, said compounds are useful for the treatment of diseases such as cancer in humans or animals and for the production of corresponding medicaments. The dosage depends on various factors such as manner of administration, species, age and/or individual state of health.

An embodiment of the invention are the compounds according to formula I for the use as pharmaceutical agents.

An embodiment of the invention is a pharmaceutical composition, containing one or more compounds according to formula I, together with pharmaceutically acceptable carriers.

Another embodiment of the invention is a medicament containing one or more compounds of formula I as active ingredients together with pharmaceutically acceptable carriers for the treatment of diseases mediated by an inappropriate activation of Aurora family kinases.

Another embodiment of the invention is a pharmaceutical composition, containing one or more compounds according to formula I, for the inhibition of tumor growth.

Another embodiment of the invention is a pharmaceutical composition, containing one or more compounds according to formula I, for the inhibition of tumor growth.

Another embodiment of the invention is a medicament containing one or more compounds of formula I as active ingredients together with pharmaceutically acceptable carriers for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is a medicament containing one or more compounds of formula I as active ingredients together with pharmaceutically acceptable carriers for the treatment of acute-myelogenous leukemia (AML, acute lymphocytic leukemia (ALL) and gastrointestinal stromal tumor (GIST).

Another embodiment of the invention is the use of one or more compounds of formula I for the manufacture of medicaments for the treatment of diseases mediated by an inappropriate activation of Aurora family kinases.

Another embodiment of the invention is the use of a compound according to formula I, for the manufacture of corresponding medicaments for the inhibition of tumor growth.

Another embodiment of the invention is the use of a compound according to formula I, for the manufacture of corresponding medicaments for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is the use of a compound according to formula I, for the treatment of acute-myelogenous leukemia (AML, acute lymphocytic leukemia (ALL) and gastrointestinal stromal tumor (GIST).

Another embodiment of the invention is the use of the compounds of formula I as Aurora A kinase inhibitors.

Another embodiment of the invention is the use of the compounds of formula I as anti-proliferating agents.

Another embodiment of the invention is the use of one or more compounds of formula I for the treatment of cancer.

Another embodiment of the invention is a pharmaceutical composition comprising a therapeutically effective amount of a compound according to formula I as active ingredients and a pharmaceutically acceptable carrier.

Another embodiment of the invention is a method of treating cancer comprising administering to a person in need thereof a therapeutically effective amount of a compound according to formula I.

Another embodiment of the invention is a method of treating colorectal cancer, breast cancer, lung cancer, prostate cancer, pancreatic cancer, gastric cancer, bladder cancer, ovarian cancer, melanoma, neuroblastoma, cervical cancer, kidney cancer or renal cancer, leukemias or lymphomas comprising administering to a person in need thereof a therapeutically effective amount of a compound according to formula I.

The compounds according to this invention and their pharmaceutically acceptable salts can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The above-mentioned pharmaceutical compositions can be obtained by processing the compounds according to this invention with pharmaceutically acceptable, inorganic or organic carriers. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

A pharmaceutical compositions comprise e.g. the following:

a) Tablet Formulation (Wet Granulation):

| Item | Ingredients | Mg/tablet | | | |
|---|---|---|---|---|---|
| 1. | Compound of formula I | 5 | 25 | 100 | 500 |
| 2. | Lactose Anhydrous DTG | 125 | 105 | 30 | 150 |
| | (direct tabletting grade) | | | | |
| 3. | Sta-Rx 1500 (pre- | 6 | 6 | 6 | 30 |
| | gelatinized starch powder) | | | | |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure:

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

b) Capsule Formulation:

| Item | Ingredients | mg/capsule | | | |
|---|---|---|---|---|---|
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure:

1. Mix items 1, 2 and 3 in a suitable mixer for 30 minutes.
2. Add items 4 and 5 and mix for 3 minutes.
3. Fill into a suitable capsule.

The following examples and references are provided to aid the understanding of the present invention, the true scope of which is set forth in the appended claims. It is understood that modifications can be made in the procedures set forth without departing from the spirit of the invention.

### EXPERIMENTAL PROCEDURES

### Example 1

### 5-(2-Methoxy-ethyl)-7,7-dimethyl-2-(5-methyl-1H-pyrazol-3-yl)-5,7-dihydro-3H-imidazo[4,5-f]indol-6-one

### i) 5,6-Diamino-1-(2-methoxy-ethyl)-3,3-dimethyl-1,3-dihydro-indol-2-one

A solution of 5,6-diamino-3,3-dimethyl-1,3-dihydro-indol-2-one (prepared according to U.S. Pat. No. 4,666,923A) (500 mg, 2.61 mmol) in anhydrous N,N-dimethylformamide (DMF) (10 ml) was treated with sodium hydride (72.6 mg, 2.87 mmol) and stirred for 1 h at room temperature. 1-Bromo-2-methoxy-ethane (259 μl, 382.5 mg, 2.61 mmol) was added dropwise. After 3 h at room temperature further sodium hydride (31.4 mg, 1.31 mmol) and 1-bromo-2-methoxy-ethane (191.2 mg, 1.31 mmol) were added and stirring continued at room temperature for another hour. Then the reaction mixture was poured into water and extracted with ethyl acetate. The combined organic phases were dried over magnesium sulfate, the solvent was removed under reduced pressure and the crude product was purified by HPL chromatography to yield 210 mg 5,6-diamino-1-(2-methoxy-ethyl)-3,3-dimethyl-1,3-dihydro-indol-2-one (32%).

### ii) 5-(2-Methoxy-ethyl)-7,7-dimethyl-2-(5-methyl-1H-pyrazol-3-yl)-5,7-dihydro-3H-imidazo[4,5-f]indol-6-one

A mixture of 5,6-diamino-1-(2-methoxy-ethyl)-3,3-dimethyl-1,3-dihydro-indol-2-one (210 mg, 0.842 mmol), 5-methyl-1H-pyrazole-3-carbaldehyde (prepared according to Tetrahedron 1995, 51(16), 4779-800; 93 mg, 0.842 mmol) and sulfur (29.7 mg, 0.926 mmol) in N,N-dimethylformamide (DMF) (6 ml) was heated at 160° C. for 65 minutes. After cooling to room temperature the reaction mixture was poured into water (40 ml). After stirring for 60 minutes at 0° C. the precipitate was filtered off, washed with water and dissolved in ethyl acetate. The aqueous mother liquid was extracted with ethyl acetate and the combined organic phases were dried over magnesium sulfate. The solvent was removed under reduced pressure und the residue dried in vacuo to yield 186 mg 5-(2-methoxy-ethyl)-7,7-dimethyl-2-(5-methyl-1H-pyrazol-3-yl)-5,7-dihydro-3H-imidazo[4,5-f]indol-6-one (65%).

MS: M=340.2 (ESI+). ¹H-NMR (400 MHz, D₆-DMSO): δ (ppm)=1.31 (s, 6H), 2.31 (s, 3H), 3.25 (s, 3H), 3.59 (t, 2H), 3.89 (t, 2H), 6.56 (s, 1H), 7.03 and 7.25 (bm, 1H), 7.35 and 7.55 (bm, 1H), 12.59 (m, 1H), 12.88 (m, 1H).

In an analogous manner as described for example 1 the following examples 2 and 3 were prepared from the appropriate starting materials:

| Example | | ¹H-NMR (400 MHz, | |
|---|---|---|---|
| No | Systematic Name | DMSO): δ (ppm) = | MS: M = |
| 2 | [7,7-Dimethyl-2-(5- | 1.37 (s, 6H), 2.32 (s, 3H), | 319.1 |
| | methyl-1H-pyrazol-3-yl)-6- | 5.02 (s, 2H), 6.59 (s, 1H), | (ESI−) |
| | oxo-6,7-dihydro-1H- | 7.22 and 7.65 (s, 1H, two | |
| | imidazo[4,5-f]indol-5-yl]- | tautomeric forms), 7.43 | |
| | acetonitrile | (m, 1H), 12.77-12.91 (m, | |
| | | 2H) | |
| 3 | 5-Allyl-7,7-dimethyl-2-(5- | 1.35 (s, 6H), 2.31 (s, 3H), | 322.0 |
| | methyl-1H-pyrazol-3-yl)- | 4.36 (d, 2H), 5.03-5.23 | (API+) |
| | 5,7-dihydro-3H- | (m, 2H), 5.89 (m, 1H), | |
| | imidazo[4,5-f]indol-6-one | 6.56 (s, 1H), 6.90 and 7.12 | |
| | | (bm, 1H), 7.38 and 7.59 | |
| | | (bm, 1H), 13.05-12.39 | |
| | | (bm, 2H) | |

### Example 4

### 7,7-Dimethyl-5-(3-morpholin-4-yl-propyl)-2-(5-trifluoromethyl-2H-pyrazol-3-yl)-5,7-dihydro-1H-imidazo[4,5-f]indol-6-one

### i) 5-Trifluoromethyl-2H-pyrazole-3-carboxylic Acid

### a) 1-Benzyl-5-furan-2-yl-3-trifluoromethyl-1H-pyrazole

To a solution of 4,4,4-trifluoro-1-(2-furyl)-1,3-butanedione (50 g, 0.240 mol) in 1M solution of hydrogen chloride in ethanol (EtOH) (24 ml, 0.024 mol) and further EtOH (520 ml) was added benzylhydrazine dihydrochloride (50 g, 0.248 mol) in small portion at room temperature. The reaction mixture was then heated under reflux for 7 h. After cooling to room temperature the reaction mixture was neutralized with saturated NaHCO₃, the EtOH was distilled off and the residual oil/water mixture was extracted with dichloromethane (300 ml). The organic phase was washed twice with water (100 ml) and dried over Na₂SO₄ and concentrated in vacuo to give 73.7 g 1-benzyl-5-furan-2-yl-3-trifluoromethyl-1H-pyrazole as a brown oil which was used crude for the next reaction.

MS: M=293.0 (API+).

### b) 2-Benzyl-5-trifluoromethyl-2H-pyrazole-3-carboxylic Acid

To a solution of 1-benzyl-5-furan-2-yl-3-trifluoromethyl-1H-pyrazole (9.5 g, 0.0325 mol) in acetone (350 ml) was added potassium permanganate (27.2 g, 0.172 mol) in water (450 ml). The reaction mixture was heated at 60° C. for 4 h. After cooling to room temperature 2-propanol (200 ml) was added and the mixture was stirred over night, it was filtered through a Celite pad and washed with acetone (1 l). The filtrate was concentrated in vacuo down to 150 ml. The residue was dissolved in 2M NaOH (20 ml) and water (150 ml). The resulting aqueous phase was washed twice with diethyl ether (70 ml) and was then acidified with 5M HCl solution (30 ml). The suspension was extracted with ethyl acetate (200 and 50 ml). The combined organic extracts were washed with water (30 ml) and brine (5 ml) and concentrated. The residue was purified by silica gel chromatography (CH₂Cl₂ with 1% acetic acid) to give 6.1 g of 2-benzyl-5-trifluoromethyl-2H-pyrazole-3-carboxylic acid (0.022 mol, 67%) as a off-white solid.

MS: M=271.1 (ESI+).

### c) 5-Trifluoromethyl-2H-pyrazole-3-carboxylic Acid

Ammonia (about 50 ml) was condensed into a three-neck-flask in an ethanol-dry ice bath and 2-benzyl-5-trifluoromethyl-2H-pyrazole-3-carboxylic acid (100 mg, 3.70 mmol) was added. To the solution sodium (about 260 mg, 11.3 mmol) was added in small portions until the blue color stayed for more then 5 minutes. The ammonia was evaporated overnight. Water was added and acidified with 2N HCl solution. The aqueous phase was extracted twice with ethyl acetate, the combined organic phases were dried over Na₂SO₄, the solvent was evaporated in vacuum to give 560 mg 5-trifluoromethyl-2H-pyrazole-3-carboxylic acid (3.11 mmol, 84%) as a yellow solid that was used without further purification.

MS: M=179.0 (API−).

### ii) 5,6-Diamino-3,3-dimethyl-1-(3-morpholin-4-yl-propyl)-1,3-dihydro-indol-2-one

### a) 3,3-Dimethyl-1-(3-morpholin-4-yl-propyl)-6-nitro-1,3-dihydro-indol-2-one

A solution of 3,3-dimethyl-6-nitro-1,3-dihydro-indol-2-one (prepared according to U.S. Pat. No. 4,666,923A; 6.3 g, 30.6 mmol) in anhydrous N,N-dimethylformamide (DMF) (40 ml) was treated with sodium hydride (0.953 g, 39.7 mmol). The resulting suspension was stirred for 1 h at 60° C. A solution of 4-(3-chloro-propyl)-morpholine (5.0 g, 30.5 mmol) in DMF (10 ml) was added dropwise. The mixture was heated to 100° C. for 10 minutes, then allowed to cool to room temperature and stirred for 1 h. After removal of the solvent the mixture was quenched with water (100 ml) and extracted with ethyl acetate (3×100 ml). The combined organic phases were dried over Na₂SO₄, evaporated and the crude product was purified by column chromatography on silica gel. Elution with ethyl acetate yielded 8.15 g 3,3-dimethyl-1-(3-morpholin-4-yl-propyl)-6-nitro-1,3-dihydro-indol-2-one (80%).

### b) 6-Amino-3,3-dimethyl-1-(3-morpholin-4-yl-propyl)-1,3-dihydro-indol-2-one

To a solution of 3,3-dimethyl-1-(3-morpholin-4-yl-propyl)-6-nitro-1,3-dihydro-indol-2-one (8.1 g, 24.3 mmol) in tetrahydrofuran (THF) palladium on charcoal was added and the mixture hydrogenated at room temperature for 4 h. After filtration and evaporation of the solvent 7.3 g 6-amino-3,3-dimethyl-1-(3-morpholin-4-yl-propyl)-1,3-dihydro-indol-2-one (99%) were isolated.

### c) N-[3,3-Dimethyl-1-(3-morpholin-4-yl-propyl)-2-oxo-2,3-dihydro-H-indol-6-yl]-acetamide

A solution of 6-amino-3,3-dimethyl-1-(3-morpholin-4-yl-propyl)-1,3-dihydro-indol-2-one (7.3 g, 24.1 mmol) in acetic anhydride (100 ml) was stirred at room temperature for 4 h. The mixture was poured into ice water and allowed to warm to room temperature. The mixture was alkalized with aqueous NaOH solution and extracted with ethyl acetate. The combined organic phases were dried over sodium sulfate and the solvent removed under reduced pressure to yield 7.8 g N-[3,3-dimethyl-1-(3-morpholin-4-yl-propyl)-2-oxo-2,3-dihydro-1H-indol-6-yl]-acetamide (94%).

### d) N-[3,3-Dimethyl-1-(3-morpholin-4-yl-propyl)-5-nitro-2-oxo-2,3-dihydro-1H-indol-6-yl]-acetamide

To a solution of N-[3,3-dimethyl-1-(3-morpholin-4-yl-propyl)-2-oxo-2,3-dihydro-1H-indol-6-yl]-acetamide (7.8 g, 22.6 mmol) in acetic acid (60 ml) nitric acid (65%, 3.2 g, 2.3 ml, 33.9 mmol) was added dropwise at 0° C. The mixture was stirred for 4 h, then poured into water. The mixture was adjusted to pH 8-9 with aqueous NaOH solution and extracted with ethyl acetate. The combined organic layers were dried over sodium sulfate and the solvent evaporated. The crude product was recrystallized from isopropanol and the concentrated mother liquid was purified by column chromatography on silica gel (ethyl acetate/methanol 9:1) to yield altogether 3.2 g N-[3,3-dimethyl-1-(3-morpholin-4-yl-propyl)-5-nitro-2-oxo-2,3-dihydro-1H-indol-6-yl]-acetamide (36%).

### e) 6-Amino-3,3-dimethyl-1-(3-morpholin-4-yl-propyl)-5-nitro-1,3-dihydro-indol-2-one

N-[3,3-dimethyl-1-(3-morpholin-4-yl-propyl)-5-nitro-2-oxo-2,3-dihydro-1H-indol-6-yl]-acetamide (3.2 g, 8.2 mmol) was dissolved in ethanol (40 ml). After addition of hydrochloric acid (25%, 4 ml, 40.8 mmol) the mixture was heated under reflux for 3 h. Most of the solvent was evaporated and water was added. The mixture was alkalized with aqueous NaOH solution and extracted with ethyl acetate. The combined organic layers were dried over sodium sulfate and the solvent evaporated. The crude product was triturated with iso-hexane and dried to yield 2.6 g 6-amino-3,3-dimethyl-1-(3-morpholin-4-yl-propyl)-5-nitro-1,3-dihydro-indol-2-one (91%).

### f) 5,6-Diamino-3,3-dimethyl-1-(3-morpholin-4-yl-propyl)-1,3-dihydro-indol-2-one

To a solution of 6-amino-3,3-dimethyl-1-(3-morpholin-4-yl-propyl)-5-nitro-1,3-dihydro-indol-2-one (2.6 g, 6.7 mmol) in tetrahydrofuran (THF)/methanol (1:1, 80 ml) palladium on charcoal (10%, 0.8 g) was added and the mixture hydrogenated at 40 mbar at room temperature for 6.5 h. After filtration and evaporation of the solvents the crude product was triturated with diethyl ether and some drops of isopropanol to yield 2.1 g 5,6-diamino-3,3-dimethyl-1-(3-morpholin-4-yl-propyl)-1,3-dihydro-indol-2-one (99%).

### iii) 7,7-Dimethyl-5-(3-morpholin-4-yl-propyl)-2-(5-trifluoromethyl-2H-pyrazol-3-yl)-5,7-dihydro-1H-imidazo[4,5-f]indol-6-one

5,6-Diamino-3,3-dimethyl-1-(3-morpholin-4-yl-propyl)-1,3-dihydro-indol-2-one (198 mg, 0.622 mmol) and 5-trifluoromethyl-2H-pyrazole-3-carboxylic acid (112 mg, 0.622 mmol) were mixed thoroughly. Polyphosphoric acid (4260 mg, 43.5 mmol) and phosphorus pentoxide (460 mg, 3.24 mmol) were added and again mixed thoroughly by spatula. The mixture was heated to 150° C. under a nitrogen atmosphere for 6 h. After cooling to room temperature the reaction mixture was quenched with ice water (20 ml) and the resulting suspension was adjusted to pH 7-8 by adding 25% aqueous ammonia. The aqueous phase was extracted with ethyl acetate, the combined organic phases were washed with water, dried over Na₂SO₄ and the solvent was evaporated in vacuo. The residue was washed with diethylether and dried in vacuum to yield 102 mg 7,7-dimethyl-5-(3-morpholin-4-yl-propyl)-2-(5-trifluoromethyl-2H-pyrazol-3-yl)-5,7-dihydro-1H-imidazo[4,5-f]indol-6-one (34%).

MS: M=463.1 (API+). ¹H-NMR (400 MHz, D₆-DMSO): δ (ppm)=1.33 (s, 6H), 1.78 (m, 2H), 2.30 (m, 6H), 3.57 (m, 4H), 3.79 (t, 2H), 7.18-7.28 (br m, 2H), 7.56-7.67 (br m, 1H), 12.98 (br, 1H), 14.64 (br, 1H).

In an analogous manner as described for example 4, step iii the following examples 5 and 6 were prepared from 5,6-diamino-3,3-dimethyl-1-(3-morpholin-4-yl-propyl)-1,3-dihydro-indol-2-one and the appropriate pyrazole-3-carboxylic acids:

| Example | | ¹H-NMR (400 MHz, | |
|---|---|---|---|
| No | Systematic Name | DMSO): δ (ppm) = | MS: M = |
| 5 | 7,7-Dimethyl-2-(5- | 1.30 (s, 6H), 1.78 (m, 2H), | 409.1 |
| | methyl-2H-pyrazol-3- | 2.31 (br s, 9H), 3.58 (br s, | (API+) |
| | yl)-5-(3-morpholin-4- | 4H), 3.75 (t, 2H), 6.56 (s, | |
| | yl-propyl)-5,7-dihydro- | 1H), 7.02 and 7.36 (br s, 1H, | |
| | 1H-imidazo[4,5- | two tautomeric forms), 7.24 | |
| | f]indol-6-one | and 7.55 (br s, 1H, two | |
| | | tautomeric forms), 12.61 (br | |
| | | s, 1H), 12.89 (br s, 1H) | |
| 6 | 7,7-Dimethyl-5-(3- | 0.95 (br s, 3H), 1.31 (br s, | 437.2 |
| | morpholin-4-yl- | 6H), 1.68 (br s, 2H), 1.78 | (API+) |
| | propyl)-2-(5-propyl- | (br s, 2H), 2.31 (br s, 4H), | |
| | 2H-pyrazol-3-yl)-5,7- | 2.50 (br s, 2H), 2.64 (br s, | |
| | dihydro-1H- | 2H), 3.58 (br s, 4H), 3.75 | |
| | imidazo[4,5-f]indol-6- | (br s, 2H), 6.58 (s, 1H), | |
| | one | 7.03-7.55 (br m, 2H) | |

### Example 7

### [7,7-Dimethyl-2-(5-methyl-1H-pyrazol-3-yl)-6-oxo-6,7-dihydro-3H-imidazo[4,5-f]indol-5-yl]-acetic Acid Ethyl Ester

To a solution of 5-methyl-1H-pyrazole-3-carboxylic acid (365 mg, 2.89 mmol), 1-hydroxybenzotriazole hydrate (535 mg, 3.49 mmol) and triethylamine (900 mg, 8.90 mmol) in N,N-dimethylformamide (DMF) (5 ml) was added N′-(3-dimethylaminopropyl)-N-ethylcarbodiimide hydrochloride (668 mg, 3.48 mmol). After 90 minutes at room temperature a solution of (5,6-diamino-3,3-dimethyl-2-oxo-2,3-dihydro-indol-1-yl)-acetic acid ethyl ester (prepared in an analogous manner as described in example 1, step i using iodo-acetic acid ethyl ester instead of 1-bromo-2-methoxy-ethane as alkylating agent; 820 mg, 2.95 mmol) in DMF (10 ml) was added and stirring continued overnight. Saturated aqueous bicarbonate solution was added and the aqueous phase was extracted three times with ethyl acetate. The combined organic phases were dried over magnesium sulfate and the solvent was evaporated. The residue was purified by silica gel chromatography (ethyl acetate). The intermediate product was then dissolved in ethanol (50 ml), treated with conc. HCl (1.75 ml) and heated under reflux for 3.5 h. Under ice cooling the reaction mixture was alkalized with saturated aqueous bicarbonate solution to pH 7-8 and most of the ethanol was evaporated. Water was added and the aqueous phase was extracted three times with ethyl acetate. The combined organic phases were dried over MgSO₄ and the solvent was evaporated. The residue was subjected to silica gel chromatography (ethyl acetate) to yield 380 mg [7,7-dimethyl-2-(5-methyl-1H-pyrazol-3-yl)-6-oxo-6,7-dihydro-3H-imidazo[4,5-f]indol-5-yl]-acetic acid ethyl ester (38%).

MS: M=368.34 (ESI+). ¹H-NMR (400 MHz, D₆-DMSO): δ (ppm)=1.20 (m, 3H), 1.34 (s, 6H), 2.31 (s, 3H), 4.15 (m, 2H), 4.60 (s, 2H), 6.55 (s, 1H), 6.94 and 7.17 (bm, 1H), 7.38 and 7.59 (bm, 1H), 12.63 (m, 1H), 12.90 (m, 1H).

### Example 8

### N-Benzyl-2-[7,7-dimethyl-2-(5-methyl-1H-pyrazol-3-yl)-6-oxo-6,7-dihydro-3H-imidazo[4,5-f]indol-5-yl]-acetamide

A mixture of [7,7-dimethyl-2-(5-methyl-1H-pyrazol-3-yl)-6-oxo-6,7-dihydro-3H-imidazo[4,5-f]indol-5-yl]-acetic acid ethyl ester (30 mg, 0.082 mmol), benzylamine (107 mg, 110 μl, 11.0 mmol) and ammonium chloride (2.5 mg, 0.047 mmol) was heated in a sealed vial under a nitrogen atmosphere to 160° C. for 3 h. After cooling to room temperature the reaction mixture was treated with water. The aqueous phase was extracted three times with ethyl acetate. The combined organic phases were dried over MgSO₄ and the solvent was evaporated. The residue was purified by HPL chromatography to yield 21 mg N-benzyl-2-[7,7-dimethyl-2-(5-methyl-1H-pyrazol-3-yl)-6-oxo-6,7-dihydro-3H-imidazo[4,5-f]indol-5-yl]-acetamide (53%).

In an analogous manner as described for example 8 the following examples 9-12 were prepared from the appropriate amines:

| Example | | ¹H-NMR (400 MHz, | |
|---|---|---|---|
| No | Systematic Name | DMSO): δ (ppm) = | MS: M = |
| 9 | 7,7-Dimethyl-2-(5- | 1.35 (s, 6H), 2.31 (s, 3H), 3.45 | 407.0 |
| | methyl-1H-pyrazol-3- | (m, 2H), 3.60 (m, 4H), 3.69 (m, | (ESI−) |
| | yl)-5-(2-morpholin-4- | 2H), 4.66 (s, 2H), 6.55 (s, 1H), | |
| | yl-2-oxo-ethyl)-5,7- | 6.89 and 7.12 (s, 1H, two | |
| | dihydro-3H- | tautomeric forms), 7.37 and 7.56 | |
| | imidazo[4,5-f]indol-6- | (s, 1H, two tautomeric forms), | |
| | one | 12.57 (m, 1H), 12.89 (m, 1H) | |
| 10 | 2-[7,7-Dimethyl-2-(5- | 1.36 (s, 6H), 2.30 (s, 3H), 4.58 (s, | 433.2 |
| | methyl-1H-pyrazol-3- | 2H), 6.54 (s, 1H), 6.94 and 7.40 | (ESI+) |
| | yl)-6-oxo-6,7-dihydro- | (s, 1H, two tautomeric forms), | |
| | 3H-imidazo[4,5- | 7.16 (m, 2H), 7.62 (m, 3H), | |
| | f]indol-5-yl]-N-(4- | 10.46 (s, 1H), 12.60 and 12.88 (s, | |
| | fluoro-phenyl)- | 2H, two tautomeric forms) | |
| | acetamide | | |
| 11 | N-(3,5-Dimethoxy- | 1.35 (s, 6H), 2.31 (s, 3H), 3.71 (s, | 489.3 |
| | benzyl)-2-[7,7- | 6H), 4.26 (d, 2H), 4.43 (s, 2H), | (ESI+) |
| | dimethyl-2-(5-methyl- | 6.36 (t, 1H), 6.44 (d, 2H), 6.56 | |
| | 1H-pyrazol-3-yl)-6- | (s, 1H), 6.99 (s, 1H), 7.48 (s, | |
| | oxo-6,7-dihydro-3H- | 1H), 8.66 (t, 1H), 12.73 (m, 2H) | |
| | imidazo[4,5-f]indol-5- | | |
| | yl]-acetamide | | |
| 12 | 2-[7,7-Dimethyl-2-(5- | 1.35 (s, 6H), 2.32 (s, 3H), 4.31 (d, | 445.2 |
| | methyl-1H-pyrazol-3- | 2H), 4.43 (s, 2H), 6.57 (s, 1H), | (ESI−) |
| | yl)-6-oxo-6,7-dihydro- | 6.94 (s, 1H), 7.16 (t, 2H), 7.31- | |
| | 3H-imidazo[4,5- | 7.35 (m, 2H), 7.53 (s, 1H), 8.72 | |
| | f]indol-5-yl]-N-(4- | (t, 1H), 12.60 (m, 2H) | |
| | fluoro-benzyl)- | | |
| | acetamide | | |

### Example 13

### 5-(2-Diethylamino-ethyl)-7,7-dimethyl-2-(5-methyl-1H-pyrazol-3-yl)-5,7-dihydro-3H-imidazo[4,5-f]indol-6-one

### i) 5,6-Diamino-1-[2-(tert-butyl-dimethyl-silanyloxy)-ethyl]-3,3-dimethyl-1,3-dihydro-indol-2-one

5,6-Diamino-1-[2-(tert-butyl-dimethyl-silanyloxy)-ethyl]-3,3-dimethyl-1,3-dihydro-indol-2-one was prepared in an analogous manner as described in example 1, step i using (2-bromo-ethoxy)-tert-butyl-dimethyl-silane instead of 1-bromo-2-methoxy-ethane as alkylating agent.

### ii) 5-[2-(tert-Butyl-dimethyl-silanyloxy)-ethyl]-7,7-dimethyl-2-(5-methyl-1H-pyrazol-3-yl)-5,7-dihydro-3H-imidazo[4,5-f]indol-6-one

5-[2-(tert-Butyl-dimethyl-silanyloxy)-ethyl]-7,7-dimethyl-2-(5-methyl-1H-pyrazol-3-yl)-5,7-dihydro-3H-imidazo[4,5-f]indol-6-one was prepared in an analogous manner as described in example 1, step ii from 5-methyl-1H-pyrazole-3-carbaldehyde and 5,6-diamino-1-[2-(tert-butyl-dimethyl-silanyloxy)-ethyl]-3,3-dimethyl-1,3-dihydro-indol-2-one.

### iii) 5-(2-Hydroxy-ethyl)-7,7-dimethyl-2-(5-methyl-1H-pyrazol-3-yl)-5,7-dihydro-3H-imidazo[4,5-f]indol-6-one

To a solution of 5-[2-(tert-butyl-dimethyl-silanyloxy)-ethyl]-7,7-dimethyl-2-(5-methyl-1H-pyrazol-3-yl)-5,7-dihydro-3H-imidazo[4,5-f]indol-6-one (790 mg, 1.80 mmol) in tetrahydrofuran (THF) (4 ml) was added a solution of tetra-N-butylammonium fluoride (1M in THF, 5391 μl, 5.39 mmol). After 1 h at room temperature the solvent was removed and the residue dissolved in ethyl acetate. The organic phase was washed with water and dried over sodium sulfate. The solvent was evaporated and the residue dried under high vacuum to yield 585 mg 5-(2-hydroxy-ethyl)-7,7-dimethyl-2-(5-methyl-1H-pyrazol-3-yl)-5,7-dihydro-3H-imidazo[4,5-f]indol-6-one which was used without further purification.

### iv) 5-(2-Bromo-ethyl)-7,7-dimethyl-2-(5-methyl-1H-pyrazol-3-yl)-5,7-dihydro-3H-imidazo[4,5-f]indol-6-one

To a solution of 5-(2-hydroxy-ethyl)-7,7-dimethyl-2-(5-methyl-1H-pyrazol-3-yl)-5,7-dihydro-3H-imidazo[4,5-f]indol-6-one (585 mg, 1.80 mmol) in dichloromethane (20 ml) were added carbon tetrabromide (1431 mg, 4.32 mmol), triphenylphosphine (1132 mg, 4.32 mmol) and triethylamine (182 mg, 1.80 mmol). After 5 h at room temperature the solvent was evaporated and to the residue was added ethyl acetate. The organic phase was washed with brine. The combined aqueous phases were extracted with CH₂Cl₂. The combined organic phases were dried over magnesium sulfate and the solvent was evaporated. The residue was subjected to silica gel chromatography (ethyl acetate/methanol 100:0->95:5->90:10) and then by HPL chromatography to yield 5-(2-bromo-ethyl)-7,7-dimethyl-2-(5-methyl-1H-pyrazol-3-yl)-5,7-dihydro-3H-imidazo[4,5-f]indol-6-one.

### v) 5-(2-Diethylamino-ethyl)-7,7-dimethyl-2-(5-methyl-1H-pyrazol-3-yl)-5,7-dihydro-3H-imidazo[4,5-f]indol-6-one

To a solution of 5-(2-bromo-ethyl)-7,7-dimethyl-2-(5-methyl-1H-pyrazol-3-yl)-5,7-dihydro-3H-imidazo[4,5-f]indol-6-one (58.5 mg, 0.15 mmol) in toluene (waterfree, 5 ml) was added diethylamine (551 mg, 7.53 mmol). After heating under reflux for 2 h the solvent was evaporated and the residue purified by HPL chromatography to yield 28.6 mg 5-(2-diethylamino-ethyl)-7,7-dimethyl-2-(5-methyl-1H-pyrazol-3-yl)-5,7-dihydro-3H-imidazo[4,5-f]indol-6-one (50%).

MS: M=379.1 (ESI−). ¹H-NMR (400 MHz, D₆-DMSO): δ (ppm)=0.89 (t, 6H), 1.30 (s, 6H), 2.31 (s, 3H), 2.50 (m, 4H), 2.63 (t, 2H), 3.76 (t, 2H), 6.56 (s, 1H), 7.06 (s, 1H), 7.46 (s, 1H), 12.55 (s, 2H).

### Example 14

### 5-(2-Amino-ethyl)-7,7-dimethyl-2-(5-methyl-1H-pyrazol-3-yl)-5,7-dihydro-1H-imidazo[4,5-f]indol-6-one

[7,7-Dimethyl-2-(5-methyl-1H-pyrazol-3-yl)-6-oxo-6,7-dihydro-1H-imidazo[4,5-f]indol-5-yl]-acetonitrile (170 mg, 0.531 mmol) was hydrogenated in 2M methanolic ammonia (20 ml) in the presence of Raney-Nickel (5 mg) for 5 h at 40 mbar. The catalyst was filtered off and the solvent evaporated. The residue was purified by HPL chromatography to yield 13.7 mg 5-(2-amino-ethyl)-7,7-dimethyl-2-(5-methyl-1H-pyrazol-3-yl)-5,7-dihydro-1H-imidazo[4,5-f]indol-6-one.

MS: M=325.2 (ESI+). ¹H-NMR (400 MHz, D₆-DMSO): δ (ppm)=1.32 (m, 6H), 1.91 (s, 2H), 2.32 (s, 3H), 2.96 (m, 2H), 3.89 (m, 2H), 6.57 (s, 1H), 7.04 and 7.57 (s, 1H, two tautomeric forms), 7.38 (s, 1H), 12.65 and 12.90 (s, 2H, two tautomeric forms).

1. A compound according to formula I, 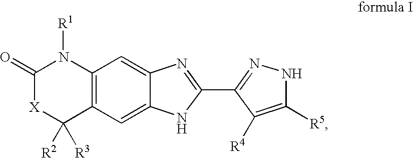
wherein,
R¹ is selected from the group consisting of:
alkyl, which is substituted once or several times by halogen, nitro, cyano, hydroxy, amino, heterocyclyl, —C(O)OH, —C(O)NH₂ or —Y—R⁶; alkenyl, which is optionally substituted once or several times by halogen, nitro, cyano, hydroxy, amino, —C(O)OH, —C(O)NH₂ or —Y—R⁶; and
alkynyl, which is optionally substituted once or several times by halogen, nitro, cyano, hydroxy, amino, —C(O)OH, —C(O)NH₂ or —Y—R⁶;
Y is selected from the group consisting of: —C(O)NH—, —C(O)N(alkyl)-, —N(alkyl)C(O)—, —NHC(O)—, —NHC(O)NH—, —NHC(O)N(alkyl)-, —NHS(O)₂—, —S(O)₂NH—, —S(O)₂N(alkyl)-, —S(O)₂—, —S(O)—, —C(O)O—, —OC(O)—, —C(O)—, —P(O)(alkyl)-, —NH—, —N(alkyl)-, —O— and —S—;
R⁶ is selected from the group consisting of:
alkyl, wherein said alkyl is optionally substituted one or several times by halogen, hydroxy, alkoxy, alkoxyalkoxy, amino, alkylamino, dialkylamino, —C(O)OH or —C(O)NH₂;
(CH₂)ₙ-aryl, wherein the aryl is optionally substituted one or several times by halogen, cyano, nitro, amino, hydroxy, (C₁-C₄)alkyl, (C₁-C₄)alkoxy, halogenated (C₁-C₄)alkyl or halogenated (C₁-C₄)alkoxy;
heteroaryl, wherein the heteroaryl is optionally substituted one or several times by alkyl;
cycloalkyl; and
heterocyclyl;
n is 0, 1 or 2;
R² and R³ are each independently hydrogen or alkyl or,
alternatively, R² and R³ together with the carbon atom to which they are attached form a cycloalkyl ring;
R⁴ is hydrogen or alkyl;
R⁵ is selected from the group consisting of: hydrogen, alkyl, halogenated alkyl, and cycloalkyl; and
X is selected from the group consisting of: a single bond, —CH₂—, and —C(alkyl)₂-;
or a pharmaceutically acceptable salt thereof.

2. A compound according to claim 1, wherein R¹ is selected from the group consisting of:
alkyl, which is substituted once or several times by cyano, amino, heterocyclyl or —Y—R⁶; and
alkenyl.

3. A compound according to claim 1, wherein:
R² is hydrogen or alkyl;
R³ is hydrogen or alkyl;
R⁴ is hydrogen;
R⁵ is alkyl or halogenated alkyl; and
X is a single bond.

4. A compound according to claim 1, wherein
Y is selected from the group consisting of: —C(O)NH—, —C(O)O—, —C(O)—, —N(alkyl)-, and —O—.

5. A compound according to claim 1, wherein
R⁶ is selected from the group consisting of:
alkyl;
—(CH₂)ₙ-aryl, wherein the aryl is optionally substituted one or several times by halogen or (C₁-C₄)alkoxy; and
heterocyclyl; and
n is 0 or 1.

6. A process for the preparation of a compound according to claim 1, comprising
reacting a compound of formula II 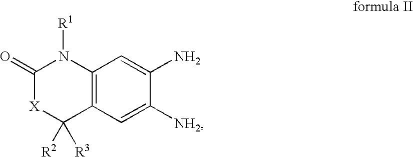
wherein:
R¹ is selected from the group consisting of:
alkyl, which is substituted once or several times by halogen, nitro, cyano, hydroxy, amino, heterocyclyl, —C(O)OH, —C(O)NH₂ or —Y—R⁶;
alkenyl, which is optionally substituted once or several times by halogen, nitro, cyano, hydroxy, amino, —C(O)OH, —C(O)NH₂ or
—Y—R⁶; and
alkynyl, which is optionally substituted once or several times by halogen, nitro, cyano, hydroxy, amino, —C(O)OH, —C(O)NH₂ or
—Y—R⁶;
Y is selected from the group consisting of: —C(O)NH—, —C(O)N(alkyl)-, —N(alkyl)C(O)—, —NHC(O)—, —NHC(O)NH—, —NHC(O)N(alkyl)-, —NHS(O)₂—S(O)₂NH—, —S(O)₂N(alkyl)-, —S(O)₂—, —S(O)—, —C(O)O—, —OC(O)—, —C(O)—, —P(O)(alkyl)-, —NH—, —N(alkyl)-, —O—, and —S—;
R⁶ is selected from the group consisting of:
alkyl, wherein said alkyl is optionally substituted one or several times by halogen, hydroxy, alkoxy, alkoxyalkoxy, amino, alkylamino, dialkylamino, —C(O)OH or —C(O)NH₂;
—(CH₂)ₙ-aryl, wherein the aryl is optionally substituted one or several times by halogen, cyano, nitro, amino, hydroxy, (C₁-C₄)alkyl, (C₁-C₄)alkoxy, halogenated (C₁-C₄)alkyl or halogenated (C₁-C₄)alkoxy;
heteroaryl, wherein the heteroaryl is optionally substituted one or several times by alkyl;
cycloalkyl; and
heterocyclyl;
n is 0, 1 or 2:
R² and R³ are each independently-hydrogen or alkyl or, alternatively, R² and R³ together with the carbon atom to which they are attached form a cycloalkyl ring; and
X is selected from the group consisting of: a single bond, —CH₂—, and —C(alkyl)₂-;
with a compound of formula IV, 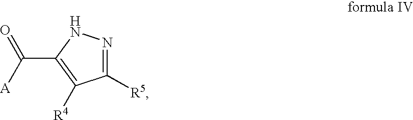
wherein
A is —OH, —Cl, —H or —OCH₃;
R⁴ is hydrogen or alkyl; and
R⁵ is selected from the group consisting of: hydrogen, alkyl, halogenated alkyl, and cycloalkyl;
to produce a compound of formula I, 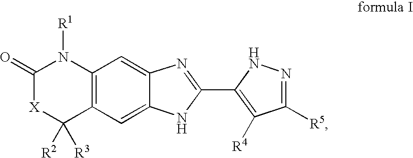
wherein R¹ to R⁵ and X are as defined above.

7. A pharmaceutical composition comprising a compound according to claim 1 and a pharmaceutically acceptable carrier.

8-10. (canceled)

11. A compound according to claim 1 selected from the group consisting of:
5-(2-Methoxy-ethyl)-7,7-dimethyl-2-(5-methyl-1H-pyrazol-3-yl)-5,7-dihydro-3H-imidazo[4,5-f]indol-6-one;
[7,7-Dimethyl-2-(5-methyl-1H-pyrazol-3-yl)-6-oxo-6,7-dihydro-1H-imidazo[4,5-f]indol-5-yl]-acetonitrile;
5-Allyl-7,7-dimethyl-2-(5-methyl-1H-pyrazol-3-yl)-5,7-dihydro-3H-imidazo[4,5-f]indol-6-one;
7,7-Dimethyl-5-(3-morpholin-4-yl-propyl)-2-(5-trifluoromethyl-2H-pyrazol-3-yl)-5,7-dihydro-1H-imidazo[4,5-f]indol-6-one;
7,7-Dimethyl-2-(5-methyl-2H-pyrazol-3-yl)-5-(3-morpholin-4-yl-propyl)-5,7-dihydro-1H-imidazo[4,5-f]indol-6-one;
7,7-Dimethyl-5-(3-morpholin-4-yl-propyl)-2-(5-propyl-2H-pyrazol-3-yl)-5,7-dihydro-1H-imidazo[4,5-f]indol-6-one;
[7,7-Dimethyl-2-(5-methyl-1H-pyrazol-3-yl)-6-oxo-6,7-dihydro-3H-imidazo[4,5-f]indol-5-yl]-acetic acid ethyl ester;
N-Benzyl-2-[7,7-dimethyl-2-(5-methyl-1H-pyrazol-3-yl)-6-oxo-6,7-dihydro-3H-imidazo[4,5-f]indol-5-yl]-acetamide;
7,7-Dimethyl-2-(5-methyl-1H-pyrazol-3-yl)-5-(2-morpholin-4-yl-2-oxo-ethyl)-5,7-dihydro-3H-imidazo[4,5-f]indol-6-one;
2-[7,7-Dimethyl-2-(5-methyl-1H-pyrazol-3-yl)-6-oxo-6,7-dihydro-3H-imidazo[4,5-f]indol-5-yl]-N-(4-fluoro-phenyl)-acetamide;
N-(3,5-Dimethoxy-benzyl)-2-[7,7-dimethyl-2-(5-methyl-1H-pyrazol-3-yl)-6-oxo-6,7-dihydro-3H-imidazo[4,5-f]indol-5-yl]-acetamide;
2-[7,7-Dimethyl-2-(5-methyl-1H-pyrazol-3-yl)-6-oxo-6,7-dihydro-3H-imidazo[4,5-f]indol-5-yl]-N-(4-fluoro-benzyl)-acetamide;
5-(2-Diethylamino-ethyl)-7,7-dimethyl-2-(5-methyl-1H-pyrazol-3-yl)-5,7-dihydro-3H-imidazo[4,5-f]indol-6-one; and
5-(2-Amino-ethyl)-7,7-dimethyl-2-(5-methyl-1H-pyrazol-3-yl)-5,7-dihydro-1H-imidazo[4,5-f]indol-6-one.
